(19) United States
(12) Patent Application Publication
(10) Pub. No.: US 2008/0131874 A1
(43) Pub. Date: Jun. 5, 2008

(54) Method for efficient post-transcriptional gene silencing using intrinsic direct repeat sequences and utilization thereof in functional genomics

(76) Inventors: Amitava Mitra, Lincoln, NE (US); Chonglie Ma, County of Pima, AZ (US)

Correspondence Address: SUITER SWANTZ PC LLO, 14301 FNB PARKWAY, SUITE 220, OMAHA, NE, 68154, US

(21) Appl. No.: 10/875,751

(22) Filed: Jun. 24, 2004

Related U.S. Application Data
(60) Provisional application No. 60/480,931, filed on Jun. 24, 2003.

(51) Int. Cl. C12Q 1/68; C12N 15/82

(52) U.S. Cl. 435/6; 435/468

(57) ABSTRACT

It is well documented that transgenes with inverted repeats can efficiently trigger post-transcriptional gene silencing (PTGS), presumably via a double stranded RNA induced by complementary sequences in their transcripts. We show here that transgenes with intrinsic direct repeats can also induce PTGS at a very high frequency (80-100%). A transgene with three or four repeats induced PTGS in almost 100% of the primary transformants, regardless of whether a strong (enhanced 35S promoter) or a relatively weak (chlorophyll a/b binding protein promoter) promoter was used. The PTGS induced by three or four repeats is consistently inherited in subsequent generations, and can inactivate homologous genes in trans. Based on the high frequency and consistent heritability, we propose that the intrinsic direct repeat within a transgene may act as a primary determinant of PTGS referred to as direct repeat-induced PTGS (driPTGS). Silencing occurred in all five genes, in this and two previous reports, suggesting that driPTGS might be a universal gene silencing mechanism both in dicotyledonous tobacco plants and monocotyledonous rice cells. In addition, driPTGS may help dissect the gene silencing mechanism and generate silenced phenotypes useful for research and plant biotechnology products.

This application is a non-provisional application which claims priority from U.S. Provisional Patent Application Ser. No. 60/480,931, filed on Jun. 24, 2003 and hereby incorporated by reference in its entirety.

### TECHNICAL FIELD

The present invention relates to the field of genetics and plant science, specifically to a method for posttranscriptional silencing of genes through the use of intrinsic direct repeat sequences.

### BACKGROUND OF THE INVENTION

Post-transcriptional gene silencing (PTGS) is a sequence specific RNA down-regulation mechanism that targets the trigger RNA molecules as well as the RNA molecules that share a certain sequence homology with the trigger. Since its first discovery in plants a decade ago, PTGS has now been characterized in a variety of eukaryotic organisms including fungi, worms, flies and mammals. A recent study even suggests that PTGS might function in bacteria. Although the name for the PTGS phenomenon differs in organisms (called ‘quelling’ in fungi, ‘RNA interference or RNAi’ in animals and ‘PTGS’ or ‘co-suppression’ in plants), it has been demonstrated recently that genes controlling quelling in fungi and RNAi in animals are homologous to genes controlling PTGS in plants. This sequence homology suggests that these processes are mechanistically linked and likely share a common ancestry.

In plants, many events can activate PTGS including high levels of transgene expression (Elmayan and Vaucheret, 1996; Lindbo et al., 1993; Que et al., 1997), concurrent expression of sense and antisense genes (Jorgensen et al., 1996; Que et al., 1998; Waterhouse et al., 1998), homology between transgenes and endogenous genes (Napoli et al., 1990; Que et al., 1997; Van der Krol et al., 1990), doublestranded RNA (Chuang and Meyerowitz, 2000; Smith et al., 2000) and special DNA arrangements (such as inverted repeats) within a transgene transcript or at a transgene locus (Hamilton et al., 1998; Stam et al., 2000). Infection of plants with some viruses such as CaMV, TBRV, TRV and PVX, activates a host PTGS-like mechanism, called virus-induced PTGS (VIGS), to eliminate the viral RNA (Al-Kaff et al., 1998; Angell and Baulcombe, 1999; Ratcliffet al., 1997). PTGS even has been triggered by introducing promoterless DNA homologous to an active endogenous gene. In most cases, however, PTGS occurs only in a portion of transformants or their progenies, suggesting that the initiation is not guaranteed and that some specific event(s) might trigger efficient gene silencing. This lack of consistency in triggering PTGS complicates the procedure of dissecting its initiation process. Therefore, methods to consistently induce PTGS are critical for understanding the PTGS process. For example, the discovery that intron-spliced hairpin RNAs induced PTGS with almost 100% efficiency clearly identifies dsRNA as a major component of the PTGS process in plants.

As part of a study on expressing multiple open reading frames in a single cistron, we serendipitously found a unique transgene-silencing phenomenon. We initially fused 2, 3 or 4 copies of the coding sequences of the cat (chloramphenicol acetyl-transferase) reporter gene into a single open reading frame with the 2A protease gene of the bovine foot-and-mouth disease virus in order to evaluate the cleavage efficiency of polyproteins mediated by 2A protease. We found that 80-100% of the primary transformants carrying tandem repeats in their transcriptional units were either completely or partially silenced. Here we report a detailed analysis of direct repeat-induced silencing in transgenic tobacco.

### SUMMARY OF THE INVENTION

It is an object of the present invention to provide for a method for efficient gene silencing through the use of intrinsic direct-repeat sequences in functional genomics.

Additional objects, advantages and novel features of the present invention will be set forth in part in the description which follows, and in part will become apparent to those skilled in the art upon examination of the following, or may be learned from practice of the invention.

### DESCRIPTION OF THE DRAWINGS

FIG. 1 is a schematic diagram of various constructs used in the method of the present invention.

FIG. 2 is a photograph of various gels of transgenes with intrinsic repeat sequences.

FIG. 3 is a photograph of Northern Blot analysis of low level steady rate mRNA in silenced plants.

FIG. 4 is a photograph of Southern Blot analysis of tDNA insertion events and a schematic diagram of constructs C3 and C4.

FIG. 5 are a series of graphs illustrating the silencing caused by transcripts with repeated sequences.

FIG. 6 are photographs of DNA gels showing the effect of gene silencing through repeated sequences.

FIG. 7 is a proposed model for driPTGS.

FIG. 8 are photographs of mutated phenotpyes observed in transgenic plants containing the GST-1 silencing locus.

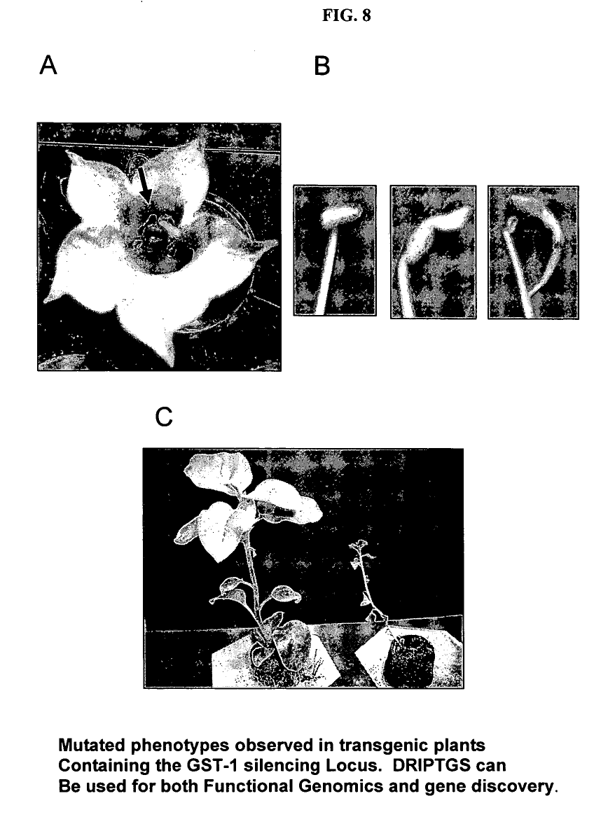

FIG. 9 are photographs of gels showing transcripts with intrinsic repeat sequences that induce high frequency gene silencing in primary transformants.

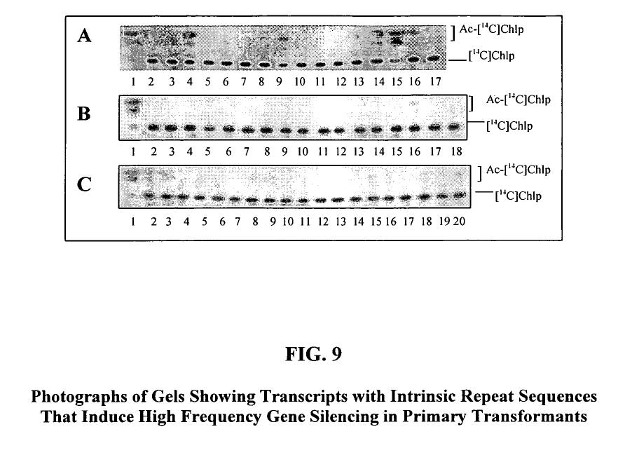

Table 1 shows the percentage of transgene silencing in primary transformants

Table 2 shows the segregation ration of non-silencing (NS)/silencing (S) plants in individuals of T₁ progeny.

### DESCRIPTION OF THE INVENTION

### Transgenes with Direct-Repeats Cause High Frequency Gene Silencing in Primary Transformants

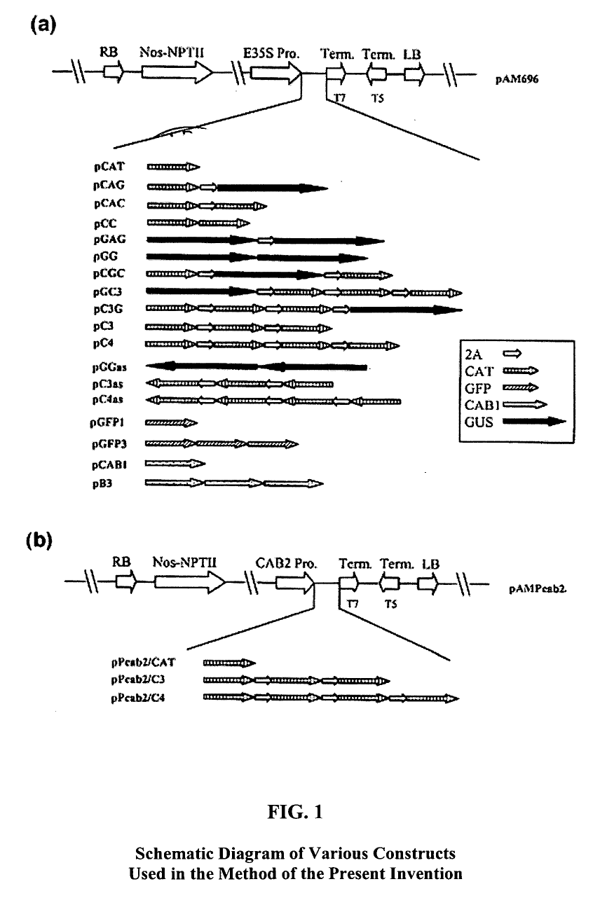

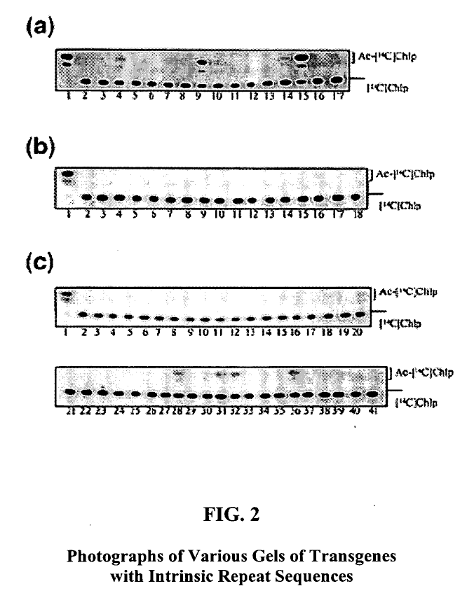

The foot-and-mouth disease virus (FMDV) 2A protease is a small protease of 16-20 amino acids that processes viral polyproteins at carboxyl termini. Translation of the viral RNA produces a polyprotein that is cleaved by the 2A protease to generate single functional protein units. We designed expression cassettes containing the FMDV 2A protease to express multiple transgenes in a single open reading frame (ORF) in plant cells. In each of these cassettes, multiple copies of reporter genes, cat and b-glucuronidase (gus) were fused into a single open reading frame with or without the 2A gene. Several expression cassettes (CC, CAC, GG, GAG, C3, C4, CGC, GC3 and C3G) contained tandemly repeated cat or gus genes (see FIG. 1). For example, construct C3 (CAT/2A/CAT/2A/CAT) had 3 copies of the cat gene arranged as a direct repeat with intervening 2A protease gene (approximately 60 base pairs) (see FIG. 1). Expression of these transgene cassettes produced non-detectable or very low enzyme activities in 80-100% of the primary transformants. Results for constructs CC, C3 and C4 are shown in FIG. 2a. High CAT activity occurred only in two (FIG. 2, panel A, lanes 9 and 15) of 15 transformants carrying construct CC (CAT/CAT). CAT activity was not detected in any of 16 transformants with construct C3 (FIG. 2, panel B, lanes 2±18). Among the 39 plants containing construct C4 (CAT/2A/CAT/2A/CAT/2A/CAT), only 4 plants (FIG. 2, panel C, lanes 28, 31, 32 and 36) had very low CAT activity, the other 35 plants lacked detectable CAT activity (FIG. 2, panel C). Individual plants with CAT activity less than 20% of the control plants were considered silenced. Thus, silencing frequency for construct C4 was deemed 100%. Similar results were obtained in plants containing other constructs, such as GG, GAG, CGC, C3G or GC3 (data not shown). Table 1 summarizes the transgene silencing frequencies of primary transformants expressing various constructs. In general, efficient silencing correlated with constructs that contained direct repeat(s); whereas silencing occurred at very low frequency in plants that expressed non-repeated sequences. Expression of a single cat gene and a gene encoding an artificial polyprotein (CAT2AGUS) resulted in 0% and 12% silencing frequency, respectively (Table 1), which are typical of our routine transformation protocols.

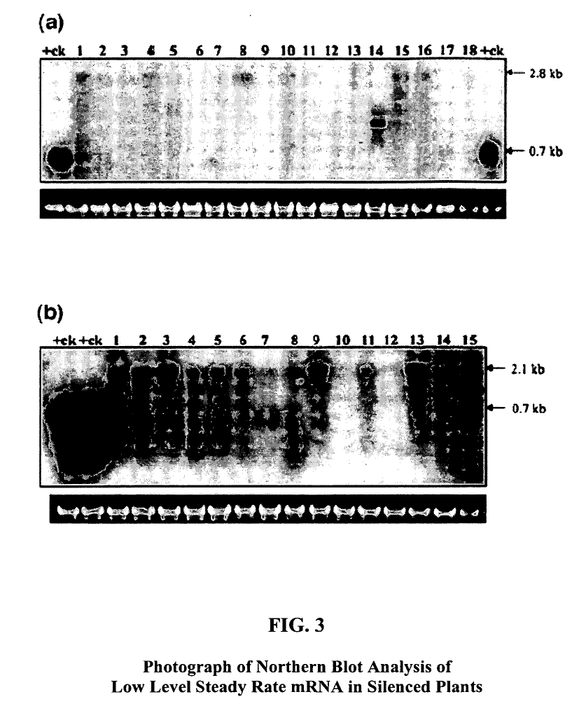

The silencing was not caused by construct errors since DNA sequencing proved that constructs CAC, C3 and C4 were correct (data not shown). Furthermore, the FMDV 2A protease gene was not responsible for silencing because deleting it from constructs GG (GUS/GUS) and CC (CAT/CAT) did not affect silencing (FIG. 2, Table 1). Northern blot analysis of total RNA from C4-transformed plants is shown in FIG. 3 (panel A). Compared to the high level of accumulation of stable mRNA detected in two plants expressing a single cat gene (FIG. 3, panel A, lanes+ck), no mRNA or very low levels of mRNA were detected in all 18 transgenic plants (FIG. 3, panel A, lanes 1-18). Although a band with a size smaller than other lines containing construct C4 was detected in one transgenic line (C4-26) (FIG. 3, panel A, lane 14), its accumulation was greatly reduced compared to control plants. The consistent correlation between enzyme activity and mRNA accumulation level suggests that the lack of mRNA reduces transgene expression. Northern blot analyses of plants containing constructs CC, CAC, GAG, or C3 produced similar results, i.e. the enzyme activities of the transgenes were proportional to the stable mRNA levels detected. Together, these data show that in all the plants with transgenes carrying direct repeats induced severe gene silencing effect. This silencing, as shown later, is at the post-transcriptional level; we refer it as direct repeat-induced PTGS, or driPTGS.

### DriPTGS is Independent of Sense or Antisense Orientation of the Repeated Sequences

Constructs CC, CAC, GG, GAG, CGC, C3G, GC3, C3 and C4 contain a long single ORF, i.e. all gene copies are fused into one reading frame (FIG. 1). We wondered if translation of repeated sequences was important for transgene silencing or if repeated sequences alone could silence. To answer these questions, we built three additional constructs: GGas, C3 as and C4 as, by inserting the GG, C3 and C4 fragments into a binary plasmid, pAM696, in an antisense orientation (FIG. 1). These three constructs were subsequently transferred into tobacco plants and production of stable, polyadenylated RNA was evaluated by Northern blot analyses of total RNA. Accumulations of stable mRNA were either undetected or greatly reduced in all 15 plants containing construct C3 as, compared to control plants expressing a single cat gene (FIG. 3, panel B, lanes 1±15 versus lanes+ck). These results showed that silencing was induced in all 15 transformants (Table 1). Similarly, silencing occurred in 100% of the plants containing constructs GGas or C4 as (Table 1). Comparison of silencing frequency induced by both sense and antisense constructs of GG, C3 or C4 revealed 100% silencing (Table 1), showing independence of orientation.

### GFP and cab1 Gene Repeats also Cause High Frequency Transgene Silencing

To determine if the silencing was general or if it resulted only from prokaryotic genes such as gus and cat, we examined two eukaryotic genes, the green fluorescent protein (GFP) gene of jellyfish Aequorea victoria and the chlorophyll a/b binding protein gene (cab 1) of Arabidopsis thaliana. Three copies of the GFP or cab1 gene were tandemly fused into one ORF to form construct GFP3 (GFP/GFP/GFP) or B3 (cab1/cab1/cab1), respectively (see FIG. 1). Northern blot analyses demonstrated that GFP mRNA accumulation was undetected or greatly reduced in 51 of 51 plants (data not shown), indicating 100% silencing (Table 1); whereas the silencing frequency was about 16% (6 out of 38) for plants expressing a single GFP gene (Table 1). Similar results were observed in 36 primary transformants expressing construct B3 and 31 primary transformants expressing a single cab1 gene (Table 1). These results suggest that transgene silencing caused by direct repeats is a general phenomenon.

### Both Strong and Weak Promoters can Induce driPTGS

Previous experiments suggested that gene silencing was often associated with the use of strong promoters such as the CaMV 35S and 19S promoters. To examine the contribution of promoter strength to silencing, we used a chlorophyll a/b binding protein (cab2) promoter from Arabidopsis to drive constructs CAT (a single cat), C3 and C4 (FIG. 1). The cab2 promoter is light-inducible and is weaker than the 35S promoter but comparable to the Agrobacterium T-DNA nopaline synthase gene (nos) promoter. Expression of a single cat gene under the control of a cab2 promoter showed strong CAT activity in all 42 primary transformants, indicating that no silencing occurred (Table 1). However, strong CAT activity was found in only one of 45 and one of 40 Pcab2-C3 and Pcab2-C4 primary transformants, respectively, indicating approximately 97% silencing frequency for both C3 and C4 constructs (Table 1). Thus the promoter strength is unimportant in determining the frequency of silencing, further suggesting that transcripts with direct repeats are efficient transgene silencing triggers.

### Analysis of T-DNA Copy Number

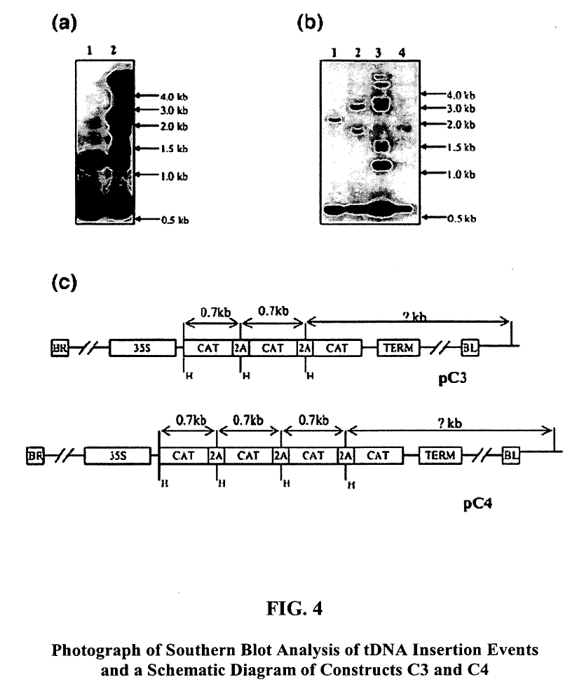

Multiple transgene copies or homology between a transgene and an endogenous gene often induce transgene silencing. Since our constructs contained repeated coding sequences, multiple insertions of T-DNA would significantly increase the number of repeated units (sequences). For example, integration of 3 or 4 T-DNA copies of construct C4 in a plant would generate 12 or 16 cat copies in a single genome. Hence, the number of T-DNA insertion events in a plant may be critical for inducing gene silencing. Southern blot analyses of HindIII digests were performed using primary transgenic plants containing C3 or C4 constructs; a typical representation of the results are shown in FIG. 4. Southern blot results revealed that the T-DNA copy number in the transgenic plants ranged from 1 to 7. Among the 5 primary transgenic lines containing construct C3, three (C3-3, C3-7 and C3-8) contained a single copy insertion of T-DNA and two plants (C3-9 and C3-10) contained multiple T-DNA insertions (FIG. 4a and data not shown). In the 12 primary transgenic lines carrying construct C4, three transgenic lines (C4-10, C4-17 and C4-26) contained a single copy of T-DNA, all others had multiple T-DNA copies (FIG. 4b and data not shown). In the transformants with a single T-DNA insertion (C3-3, C3-7, C3-8, C4-10, C4-17 and C4-26), the cat gene was silenced as efficiently as in transformants with multiple T-DNA insertions (FIG. 2, panels B and C). Furthermore, these plants used for Southern blots were all primary transformants (e.g. hemizygous). These results suggest that silencing caused by direct repeats was independent of that caused by repeated T-DNA copies.

### Silencing Caused by Three or More Repeats is Consistently Inherited Whereas Silencing Caused by Two Repeats is Often Reversed

Reversion (loss of silencing) often occurs in progeny of silenced plants. T1 seedlings derived from silenced and non-silenced primary (T0) transgenic plants were sampled at two ages for continued transgene activity as described in the experimental procedures.

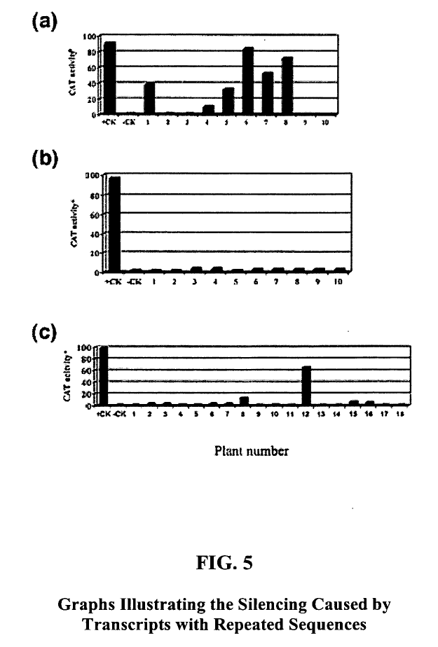

Ten T1 seedling pools derived from silenced CAC lines were tested for CAT activity. Five lines retained their silencing state in all T1 seedlings, but partial or complete reactivation of the silenced transgene was observed in 5 lines (CAC-8, CAC-19, CAC-20, CAC-21 and CAC-23) (FIG. 5a, lanes 1, 5-8). Eighteen individuals from line CAC-8 were transferred to the greenhouse and, 1 week before flowering, protein extracts were assayed for CAT activity.

Silencing was reversed in 6 of the 18 transformants (see Table 2), suggesting high reversion frequency. In contrast lines carrying constructs C3 or C4 were stable. No reversion occurred in 10 seedling pools of C3 lines (FIG. 5b) and partial reactivation of the transgene only occurred in one (line C4-24, FIG. 5c, lane 12) of 18 C4 lines (FIG. 5C). Analysis of the 18 C4-24 T1 individuals later in development (1 week before flowering) showed that silencing was restored in all these plants (Table 2), indicating that the reversion of these seedlings was temporary. Also, all 18 individuals from every C3-1, C3-3, C3-12, C4-1, C4-11 or C4-16 transgenic line, assayed at the same stage as line C4-24, were in the silenced state (Table 2). In C4 primary transformants, the transgene was not completely silenced in 4 lines (C4-26, C4-29, C4-30 and C4-34) (FIG. 2c, lanes 28, 31, 32, 36). However, CAT assays of their progeny seedling pools revealed that the T1 seedlings of all four lines (C4-26, C4-29, C4-30 and C4-34) became silenced (data not shown). Not only was silencing stably inherited, but it was also enhanced in the T1 progeny from these transgenic lines. In addition, the transgene was silenced in all T2 plants of several C4 transgenic lines (C4-1, C4-11, C4-16 and C4-21) (data not shown).

CAT activity was detected in only half (9 out of 18) of T1 plants from a non-silencing CAC line (CAC-4) (Table 2). Such a high frequency segregation of silenced plants in the T1 generation was not, however, observed in plants containing a single cat or gus gene (FIG. 1). No silencing occurred in the 18 and 40 T1 plants of transgenic lines CAT-1 and GUS-1, respectively (Table 2).

An Approximate 25 Nucleotide RNA Species was Detected in All Silenced Plants Examined, Indicating Involvement of a Post-Transcriptional Silencing Mechanism

The repeat-induced silencing leads to several questions: (i) How does it occur? (ii) Is it at the transcriptional level or post-transcriptional level? and (iii) Why does it occur so efficiently? To begin to answer these questions, we used a strategy developed by Hamilton and Baulcombe (1999), who discovered a small RNA species (21-25 nucleotides long) associated with post-transcriptional gene silencing (PTGS). Similar small RNAs of 21-23 nt were found in ds-RNA-induced PTGS in animal cells, called RNA interference (RNAi).

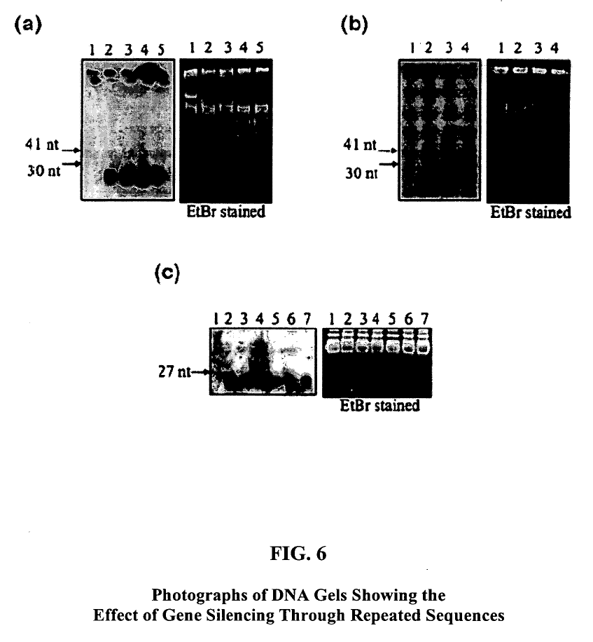

To search for the small RNA in our silenced plants, we conducted Northern blot analyses on both primary transformants and T1 plants (FIG. 6). The approximate 25 nt small RNA was detected in all 7 silenced T1 plants from independent C4 transgenic lines (FIG. 6a, lanes 2-5; FIG. 6b, lanes 2-4) but not in a control plant that expressed a single cat gene (FIG. 6a,b, lane 1). The approximate 25 nt RNA was also present in 6 independent primary transformants that expressed construct B3 (cab1/cab1/cab1) (FIG. 6c, lanes 2-7), but not in a control plant with a non-silenced, single cab1 gene (FIG. 6c, lane 1). Small RNA was also detected in a silenced T1 plant (CAC-3-4) but not in a non-silenced T1 plant (CAC-3-3) (data not shown). Since we used probes generated from ds-DNA fragments, we could not distinguish whether the small RNA was sense or antisense. We assume that the small RNA included both sense and antisense species as observed by Hamilton and Baulcombe (1999). Nevertheless, the presence of approximate 25 nt RNAs in all silenced plants strongly suggests that the silencing mechanism activated by direct repeats is post-transcriptional.

### Silencing Caused by Direct-Repeats can Inactivate Homologous Genes in Trans (Co-Suppression)

It is well established that PTGS involves sequence specific RNA degradation. To test whether the PTGS, induced by transcripts with tandem repeats, can silence the homologous gene in trans, we carried out two sets of reciprocal crosses using a non-silenced line, CAT-2, that expressed a single cat gene and two silenced lines, C4-7 and C4-10, that contained construct C4. CAT activities of the F1 plants from each of the 4 crosses (CAT-2 3 C4-7, C4-7 3 CAT-2, CAT-2 3 C4-10 and C4-10 3 CAT-20) demonstrated that whenever a CAT-2 locus was present with a C4-7 or C4-10 locus, CAT activity was absent or significantly reduced, indicating that both the C4-7 and C4-10 loci could inactivate the non-silenced CAT-2 locus in trans. To further investigate trans inactivation of homologous genes induced by direct repeats we used double transformations. First, a hygromycin resistant transgenic line (779CAT-3) that carried a single cat gene under the control of 35S promoter was generated (data not shown). Second, this line was transformed again with plasmid pC3 that contained construct C3 and an nptII gene (FIG. 1). The 779CAT-3 line was also transformed with another single cat gene (PCAT, FIG. 1) as a control. The double transformants were selected on medium containing both hygromycin and kanamycin. Analyses revealed that CAT activity was eliminated or significantly reduced in 18 of 22 double transformants, suggesting a cosuppression frequency of >80%, which was not observed in control double transformants that contained two cat loci. CAT activity was reduced in only 1 out of 13 control double transformants. Southern blot analyses showed that a single copy of construct C3 was often sufficient for co-suppression. These data further demonstrate that the PTGS caused by direct repeats can efficiently trans-inactivate an homologous gene.

### Intrinsic Direct-Repeat Causes Consistent, Posttranscriptional Transgene Silencing

A phenomenon, similar to driPTGS observed in this study, has previously been implied in two previous studies. In an attempt to analyse the roles of transgenes on RNAmediated virus resistance, Sijen et al. (1996) used the movement protein (MP) gene of cowpea mosaic virus (CPMV) to build constructs with various configurations, including sense, antisense, inverted repeat and tandem repeat. They found that 60% of transgenic plants carrying the MP tandem repeat construct were resistant to CPMV, whereas only 20% and 5% of transgenic plants with a single sense MP gene or an inverted repeat of MP gene sequences, respectively, were resistant. In another study, Wang and Waterhouse (2000) reported that when two transgenic rice callus lines with stable GUS expression were transformed again with a tandemly repeated gus construct, GUS was co-suppressed in 54% of doubletransformants. This frequency was substantially higher compared to frequencies of 21% or 19-34% when doubletransformation used antisense or sense gus constructs, respectively. However, with their limited data, neither of these two groups explained this phenomenon.

In this study, we have shown that direct-repeat sequences induce severe gene silencing in transgenic tobacco plants. Through a series of experiments, several remarkable characteristics of this direct repeat-induced PTGS (driPTGS) have been revealed. The first striking feature of driPTGS is high frequency and consistency. Transgenes were silenced in 80% to 100% of the primary transformants that carried direct repeats (Table 1). In particular, transgenes containing three or four direct repeats were silenced in almost 100% of transformants regardless of the presence of a strong (enhanced 35S promoter) or a relatively weak (chlorophyll a/b binding protein gene promoter) promoter (Table 1). High silencing frequency occurs in all transgenes that carry direct repeats no matter whether the repeat is present throughout a transcriptional unit (constructs CC, CAC, GG, GAG, C3, C4, B3 and GFP3), is located at 5′ (C3G) or at 3′ (GC3), or is interrupted (CGC) (FIG. 1 and Table 2). Separation of two cat genes (approximately 0.7 kb each) with a 1.8-kb gus gene in construct CGC (FIG. 1) did not prevent repeat-induced gene silencing (Table 1, CGC versus CC and CAC). Analyses of steady-state mRNA show that failure to accumulate transgene mRNA is a characteristic feature of every silenced plant (FIG. 3).

Detection of the small (21±25 nt) RNA species, which is considered to be a marker for PTGS, in all the silenced plants suggests a PTGS mechanism. Since no endogenous homology was involved in this study, the direct repeats within a transcription unit were the sole cause of the transgene silencing. Moreover, high frequency driPTGS was induced in five independent genes, cat, cab1 and GFP, gus and CPMV movement protein genes, suggesting that driPTGS is a universal mechanism.

Another striking feature of driPTGS is the inherited stability of the silencing. PTGS is often unstable as reversion often occurs in the progeny of silenced plants. When interacting loci are involved in silencing, such as genetic crosses, presence of multiple copies, or cosuppression, reversion of the silenced status occurs as the interacting loci segregate or copy number is reduced. In other instances, silenced transgenes are temporarily reactivated during specific developmental stages. As our data show that silencing induced by three or four direct repeats is independent of transgene copy number, we predict that driPTGS should be stably inherited.

Indeed, silenced phenotype was maintained in progenies of all lines containing three or four repeats (FIG. 5 and Table 2). The exception is line C4-24 that showed reactivation of the transgene during the seedling stage (FIG. 5c, lane 12) but reverted back to silenced phenotype at later developmental stages (Table 2, row C4-24). A similar temporary reversion phenomenon was reported by Elmayan and Vaucheret (1996). However, reactivation often occurred in progeny of CAC plants with two direct repeats (FIG. 5c and Table 2). In this case, gene dosage may be involved as discussed later. Nevertheless, the almost 100% silencing frequency in plants containing three or four repeats and stable inheritance of silenced phenotype in progeny of these plants clearly demonstrated that transgenes with direct repeats can induce consistent, post-transcriptional gene silencing.

### Is RNA with Direct Repeats a Primary Determinant of PTGS?

Various models have been proposed to explain PTGS under diverse circumstances. These models are classified into three categories: threshold models, aberrant RNA models and ectopic interaction models. Threshold models evoke a mechanism to sense the quantitative abnormality of a specific RNA species (e.g. too much RNA), whereas aberrant RNA models evoke a mechanism to recognize the qualitative abnormality of a specific RNA species (e.g. repetitiveness, double stranded, or without polyadenylation). The third model assumes an ectopic pairing of DNA-DNA, RNA-DNA or RNA-RNA. Whether driPTGS fits in any of these models or is distinct remains undetermined. Based on our data, we suggest a model for PTGS induction by direct repeats.

Recently, researches from different systems, including plants, Caenorhabditis elegans and Drosophila, identified dsRNA as a common link for PTGS triggered by various events (for review, see Vance and Vaucheret, 2001). In cases where dsRNA could form directly, for example, transgenes carrying intrinsic inverted repeats (i/r), and transgenes arranged as i/r, the triggering of the PTGS process may be straightforward. Presumably, the dsRNA molecules initiate a mechanism resembling dsRNA-induced RNA interference (RNAi) that is well documented in C. elegans and Drosophila. It is known that in the RNAi process, dsRNA is first chopped via RNase IIIrelated enzymes such as Dicer, into small interfering RNAs (siRNAs) of both polarities. The siRNAs then actuate the corresponding mRNA degradation in two different ways, as suggested by Hammond et al. (2000) and Lipardi et al. (2001). The siRNAs either guide a nuclease complex, called the RNA-induced silencing complex (RISC), to degrade mRNA, or antisense siRNAs act as primers for synthesis of complementary strands on mRNA templates. In the latter case, new dsRNAs are generated, resulting in production of more siRNAs, thus RNAi is maintained and amplified.

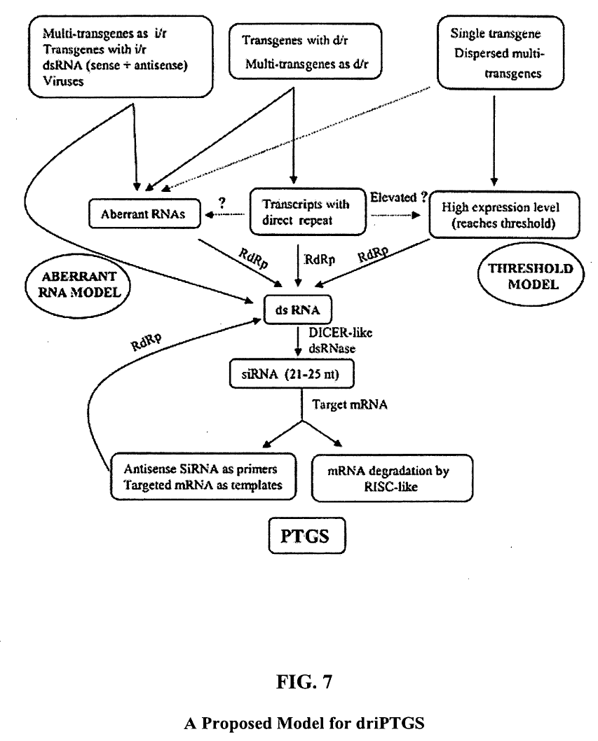

However, what initiates the production of dsRNA is unknown except where PTGS is triggered by ds-RNA. This is partially because such a process is difficult to demonstrate experimentally. Lack of consistency (often a portion of transformants and their progenies exhibit PTGS) further complicates understanding of the process. In contrast, our study shows that transgenes with direct repeats induce PTGS as efficiently as dsRNA. This suggests that RNA products (mRNA, aberrant transcripts or breakdown products of mRNA) derived from these transgenes might be templates for an RNA-directed RNA polymerase (RdRp) complex. In this scenario, there are two potential pathways for driPTGS, as shown in our proposed model (FIG. 7). Between these two possibilities, we prefer the first one, which assumes that transcripts with direct repeats (d/r) are the primary determinants of PTGS, e.g. d/r transcripts alone trigger PTGS. That all transgenes with three or four direct repeats induced silencing in almost 100% of transformants, independent of promoter strength (Table 1), and stable inheritance of silencing (Table 2) support the assumption. However, the fact that (i) transgenes with two direct repeats, such as transgenes CC, CAC, CGC, GAG (FIG. 1) and transgenes described by Sijen et al. (1996) and Wang and Waterhouse (2000), induced PTGS at lower frequencies than transgenes with 3 and 4 repeats (Tables 1) and (ii) silencing in progenies of plants of transgenes with 2 repeats was less stable than in plants of transgenes with 3 or 4 repeats (Table 2), is puzzling. Why transcripts with 2 repeats can trigger PTGS in some plants but not in others is difficult to explain. Possibly the number of repeats determines efficiency of PTGS triggering, e.g. a dosage effect. Transgenes with 2 repeats are capable but inefficient triggers. This is analogous to the observation reported by Smith et al. (2000) that transgenes with inverted repeats (i/r) could not induce PTGS in 100% of transgenic plants until an intron was inserted between the i/r sequences, presumably intron splicing promotes formation of perfect dsRNA.

The second driPTGS pathway (in the aberrant RNA class) assumes that aberrant RNAs (premature transcripts, breakdown products or antisense RNA) from transgenes activate an RdRp complex to synthesize an antisense strand on the aberrant RNA template, producing a dsRNA product. This model reduces the conflict mentioned in the first pathway since a transgene with more repeats may produce more aberrant RNAs. However, what property of an aberrant RNA activates the RdRp complex remains unknown. Moreover, whether expression of transgenes with direct repeats produces aberrant RNA in such high frequency (80-100% of transgenic plants) is questionable. Alternatively, driPTGS may be triggered by d/r transcripts themselves as aberrant RNA, even if they are perfect mRNA and translatable. driPTGS also fits the threshold model. In this case, repeats may be more efficient than single copies in triggering silencing. Nevertheless, our phenomenon sheds new light on PTGS. Further study of its mechanism will improve the understanding of PTGS, especially processes upstream of the dsRNA step. As shown in the model (FIG. 7), a crucial component for these upstream steps is a cellular RdRp complex, which has been proposed by Vance and Vaucheret (2001) to consist of products of genes that are required for PTGS, such as SGS2 (a RdRp), SGS3 (a coiled coil protein) (Mourrain et al., 2000), AGOl (a PAZ/Piwi protein) (Fagard et al., 2000) and SDE3 (an RNA helicase) (Dalmay et al., 2001). If such a complex exists, one of the RNA products (mRNA, breakdown products or aberrant RNA) derived from transgenes with direct repeats will be a preferred substrate for the complex. This may provide an ideal system for dissecting PTGS mechanisms. Moreover, the high frequency of co-suppression induced by transgenes with d/r (Wang and Waterhouse, 2000; this study) indicates a potential powerful tool for functional knockouts of endogenous plant genes.

### Experimental Procedures

### Transgene Constructs

All the transgenes listed in FIG. 1 were built using PCR. To facilitate cloning procedures, a restriction enzyme site was added to every synthesized oligonucleotide primer. Different PCR fragments were first assembled on a cloning vector, pGEM-7z (Promega, Madison, Wis., USA), to form all the constructs as shown in FIG. 1. Subsequently, all these constructs were transferred into a binary vector, pAM696 (Mitra, unpublished) or pAMPcab2 (this study). A common feature of these constructs is that multiple copies of genes in a certain construct were fused into single open reading frame. For example, construct GFP3 consists of three directly repeated GFP (green fluorescent protein) genes. The first two gene copies do not have stop codons and were joined by the third gene copy in frame so that expression of this GFP3 construct leads to a polyprotein with three GFP ORFs fused together (For details, see FIG. 1).

### Plant Material and Transformation

Leaf discs of Nicotiana tabacum var. Xanthi-nc (abbreviated as XNC) were transformed using protocols described by Horsch et al. (1985) with minor modifications. Green shoots, approximately 1 cm long, were excised from transformed calli and transferred to MS medium supplemented with kanamycin and carbenicillin (200 mg 1-1 of each) for rooting. Plantlets with sound roots were then transplanted onto Jiffy-7 peat pellets (Jiffy Products of America Inc., Batavia, Ill., USA) and cultured for 2 weeks. Transgenic plants were eventually planted in a sterile planting soil mixture in clay pots and grown in the greenhouse. Seeds of transgenic plants were harvested separately for each individual. For double transformations, a primary transformant that had a single copy of cat gene and a hygromycin resistance gene, was first generated and subcultured. Subsequently, the progenies were transformed with either pCAT or pC4 (FIG. 1), and transformants were selected on MS medium supplemented with 150 mg 1-1 kanamycin and 35 mg 1-1 hygromycin.

### Enzyme Assays

All GUS staining procedures were as described by Jefferson (1987). CAT assays were as described by Gorman et al. (1982).

Investigation of Reversion of Silencing in T1 Progenies

Primary transformants (the T0 generation) were self-fertilized. The seeds were sterilized and plated on MS medium supplemented with 200 mg 111 kanamycin. About 100 seedlings, derived from a given T0 plant, were pooled and assayed for CAT. Eighteen seedlings for every selected T1 progeny line were transferred to the greenhouse and assayed for CAT before flowering.

### Molecular Characterization

To isolate RNA, tobacco leaves (0.2 g) were ground to fine powder in liquid nitrogen and subsequently extracted with a (1:1 v/v) Trizol Reagent (Gibco BRL, Grand Island, N.Y., USA) (0.6 ml) and chloroform mixture. After a short spin, total RNA was precipitated from the supernatant with an equal volume of iso-propanol. For isolating small RNAs, a lithium chloride (2 M) precipitation procedure was added to remove high-molecular weight RNA as described by Llave et al. (2000). To detect steady-state RNA molecules, approximately 20 mg total RNA from each sample was separated on a 1.2% agarose gel, transferred onto Zeta membrane (Bio-Rad, Hercules, Calif., USA) and the membrane was subsequently hybridized with 32P-labelled probe at 65° C. overnight as described by the vendor. To detect small RNA molecules, 50-60 mg RNA for each sample was fractionated on a 15% TBE-PAGE gel with 7.0 M urea (Bio-Rad), transferred onto Zeta membrane (Bio-Rad) and probed by hybridization as described by the vendor except that hybridization was done at 50° C. for 36 h.

### Further Tests and Results

Posttranscriptional gene silencing (PTGS) and co-suppression in plants, quelling in fungi and RNAi in animals are now known to be mechanistically related. A common characteristic of these processes is the sequence-specific RNA degradation. New tools based on this characteristic have been developed for both plant and animal systems. Thanks to these methods, it is now possible that virtually any gene can be functionally knocked-out in some plant species, cultured human cells, and some animals, such as C. elegans and Drosophila, in a matter of several weeks. For example, a system developed by Baulcombe's group (Ratcliff et al., 2001, Plant J. 25, 237-245) uses TRV (tobacco rattle virus) as trigger to initiate a PTGS-like process, called virus induced gene silencing or VIGS. As a consequence of VIGS, TRV viral RNAs and host RNAs (if any) homologous to viral RNAs are specifically degraded. In this case, a fragment of a host gene inserted into the viral RNAs will make mRNAs of gene(s) homologous to the fragment become the target of VIGS, resulting in loss of function of the endogenous gene(s). This system allows quick and easy functional analysis of a gene. However, the host-dependence is its limitation, e.g., efficient VIGS depends on viral infection.

In our previous work, we characterized a unique transgene silencing phenomenon, referred to as direct repeats induced posttranscriptional gene silencing or driPTGS. A remarkable feature of driPTGS is that transgenes with three or more direct repeats can induce high frequency, consistent PTGS. In this study, we used this feature to develop a driPTGS-based system for functional analysis of plant genes.

We previously mentioned that silencing induced by direct-repeats (driPTGS) could inactivate homologous genes in trans (co-suppression). When a transgenic tobacco line expressing a single copy CAT gene was transformed again by a transgene carrying three direct-repeats of CAT gene (pC3), expression of the single copy CAT gene was completely or partially suppressed in more than 80% of independent double transformants (18 out of 22). Such a high suppression frequency suggests that driPTGS could be developed as a research tool to efficiently turnoff or down-regulate expression of plant genes. To explore this possibility, we further investigated these 22 double transformants in details and examined more constructs carrying direct repeats. FIG. 1 shows a list of various constructs used in this study.

### Three Direct-Repeats of CAT Gene, Regardless Whether in Sense or Antisense Orientation, Efficiently Suppressed an Unlinked CAT Transgene

We used the double transformation approach as described previously. We previously generated two hygromycin-resistant tobacco lines, 779CAT-3 and 779GUS-1, which carried a single, highly expressed CAT or a GUS gene, respectively. Subsequently, 779CAT-3 was transformed again with constructs carrying either three repeated CAT genes (pC3) or a single CAT gene (pCAT). The resulting double transformants were named as C-C3-1 to -22 and C-C3-1 to -13, respectively. CAT enzyme activity analyses of C-C3 and C-CAT lines are shown in FIG. 2. The high level CAT activity of 779CAT-3 was eliminated or greatly reduced in 18 out of 22 C-C3 lines, indicating an 82% cosuppression frequency. However, such high frequency was not observed when another single CAT gene (PCAT) was introduced into 779CAT-3. CAT activity was significantly reduced in only one out of 13 double transformants, an 8% cosuppression frequency. For comparison, we used an antisense method, a strategy often used to obtain high frequency cosuppression, to suppress the ‘endogenous’ CAT gene in 779CAT-3. The cosuppression frequency, 33%, induced by antisense CAT gene (pCATas) was still much lower than that (82%) induced by direct repeats (pC3). To confirm the cosuppression, Northern blot analyses of all 22 C-C3 double transformant lines were carried out. Results showed that both CAT and C3 mRNAs were either eliminated or significantly reduced in all 18 silenced lines (C-C3-1 to-8, -10 to -16, -18, -21 and -22) but not the 4 lines (C-C3-9, -17, -19 and -20) shown high CAT activity. To investigate the correlation between C3 copy number and silencing, we performed Southern blots on all 22 C-C3 lines. Results showed that 12 out of 22 double transformants carried a single integration of C3. Among these 12 single-copy lines, cosuppression was efficiently induced in 8 lines, suggesting that a single copy of transgene with direct repeats is often sufficient to induce silencing. It is worth to mention that all 4 lines without cosupression (C-C3-9, -17, -19 and -20) were among these 12 single-copy lines, indicating that co-suppression occurred in all double transformants with multiple copies of construct with three direct CAT repeats (pC3). In these 4 lines without co-supression, the steady mRNA of repeated transgene (pC3) was hardly detectable, whereas the steady mRNA of single CAT gene (pCAT) remained at high level. Apparently, pC3 but not pCAT was silenced in these 4 lines, presumably due to a transcriptional gene silencing effect. Detection of siRNAs, a hallmark of PTGS, in suppressed plants but not in non-suppressed plants indicated that the cosuppression was at the posttranscriptional level.

To eliminate the possibility that high frequency cosuppression might be caused by DNA with direct repeats alone and not by transcripts with direct repeats, we transformed line 779CAT-3 with two control constructs pC3/NP and pC4/NP which contained 3 and 4 direct repeats of CAT gene but no promoter, respectively. These 3 and 4 repeats would not be transcribed in plant cells because of the lack of promoter to initiate transcription. Cosuppression was not detected in any of the C-C3/NP and C-C4/NP double transformants, suggesting that cosuppression is caused solely by transcripts with direct repeats, but not by DNA with direct repeats. Moreover, introduction of a construct carrying three repeats of the CAT gene in an antisense orientation (pC3 as) into 779CAT-3 also induced a cosuppression frequency of 80%, indicating that PTGS can target both the trigger and its antisense RNA. Together, these data demonstrate that transgenes with direct repeats in either sense or antisense orientation can be used to induce cosuppression at very high frequency. This is consistent with our previous observation that transgenes with three or more direct repeats induced gene silencing in almost 100% of primary transformants.

### GUS Transgene Fused Downstream of Three CAT Repeats Induces Silencing of an Initially Highly Expressed, Unlinked GUS Transgene:

Our previous results showed that construct pC3G, which carried a GUS gene linked to three repeated CAT gene fragments, also induced gene silencing in almost 100% primary transformants and siRNAs corresponding to GUS gene were detected in silenced C3G. We reasoned that same as direct repeats, a non-repeated fragment linked to direct repeats would also induce efficient suppression of expression of genes homologous to the fragment. To test this assumption, we introduced construct pC3G and control construct CAG into 779GUS-1 tobacco plants via Agrobacterium transformation. The resulting double transformants were referred to G-C3G-1 to -37 and G-CAG-1 to -39, respectively. GUS assays of double transformants demonstrated that the strong GUS expression of 779GUS-1 was eliminated or significantly reduced in 84% (31 out of 37) of G-C3G double transformants. In contract, reduction of GUS expression occurred only in 36% (14 out of 39) of G-CAG double transformants. The only difference between constructs pC3G and pCAG was that C3G carried two extra copies of CAT genes in its 5′ coding region. Apparently, these two extra copies of CAT gene gave construct pC3G a 133% increase (from 36% to 84%) in terms of co-suppression frequency of GUS gene, indicating that fragments linked to direct repeats have the same efficiency in co-suppression as direct repeats themselves.

### Transitive Silencing

It is known that silencing is capable to spread in both directions (e.g from 3′ to 5′ and from 3′ to 5′). To take this spread-of-silencing feature one step further, we crossed two double transformants G-CAG-4 and -5, both containing non-silenced GUS and CAG loci, to a silenced C4 loci (C4-10). GUS activity analyses showed that 9 out of 26 F1 plants of [C4-10×G-CAG-4] and 8 out of 24 F1 plants of [G-CAG-5 X C4-10] had strong GUS activity. The rest had either very weak or no detectable GUS activity (data not shown). To find out the correlation between transgenic loci and GUS phenotype, we further conducted Southern blot analyses on these plants. Results showed that all plants with strong GUS activity had one of the following genotypes, a GUS or CAG locus alone, a GUS locus plus either a CAG or C4 locus (data not shown), indicating that GUS gene expression was not affected by non-silenced CAG locus and the silenced C4 locus in these cases. However, when a GUS locus co-existed with both CAG and C4 loci, e.g. a GUS(+)CAG(+)C4(+) genotype, GUS expression was repressed in all 13 plants (6 from cross [C4-10 X G-CAG-4] and 7 from cross [G-CAG-5 X C4-10]) (data not shown). In this case, although there is no homology between C4 and GUS loci, silencing initiated by transcripts with 4 direct CAT repeats (pC4) is still passed to GUS transcripts via ‘bridge’ transcripts CAG, which share homology with both C4 and GUS transcripts. Detection of high levels of the ˜21/25 small RNA (siRNA) species using probes derived from both 3′ and 5′ GUS sequences further confirms that silencing of GUS gene expression was caused by silencing passed from C4 transcripts. Collectively, our data suggest that not only is the driPTGS triggered by C 4 transcripts (primary silencing) spreads to the entire target CAG transcripts (e.g. from CAT gene area to GUS gene area), but also the silencing-spread (secondary silencing) is capable of suppressing a third locus (GUS).

Transgenes with direct repeats in either sense or antisense orientation induce co-suppression at very high frequency. Transgenes linked to direct repeats of non-homologous sequences is also capable of inducing high frequency co-suppression.

Although whether driPTGS preferentially targets direct repeats or not caused by introduction of another GUS copy (C3G), but by the three CAT copies linked to the GUS. Apparently, silencing initiated by d/r was spread to the whole transcript. These data suggest that it might be possible to target endogenous gene expression just by fusing an endogenous sequence fragment to a generic d/r vector as is proposed in the Experimental Plans. It is important to note that the advantage of d/r-silencing over hairpin silencing would also come from the fusion strategy, as there would be no need to create complicated arrays of sequences organized either in inverted (hairpins) or direct repeats.

### Materials and Methods for Further Tests & Results

### Plant Materials and Transformation

Two hygromycin-resistent transgenic tobacco lines 779GUS-1 and 779CAT-3 carried a single highly-expressed GUS or CAT gene, respectively. Both GUS and CAT gene were under the control of CaMV 35S promoter. These two lines were subsequently transformed again (double transformation) by a various constructs (pC3, pCAG, pC3G, pCAT or pCATas, etc.). Because all constructs used for second transformation contained a NPTII gene for plant selection, regeneration and growth of double transformants were carried out on MS medium supplemented with both hygromycin (30 mgL-1) and kanamycin (150 mgL-1). Transgenic line C4-10, which contained a single copy of construct pC4 (4 repeats of CAT gene coding region in a single open reading frame), was generated in our previous work (Ma and Mitra, 2002). Double transformant lines G-CAG-4 and G-CAG-5 were generated in this study by transforming transgenic line 779GUS-1 with construct pCAG. Each line contained a single copy of GUS and CAG transgenes, respectively. They normally expressed both GUS and CAG loci.

### Transgene Constructs

Constructs pCAT, pC3, pC3 as, pC3G and pCAG were described in details in our previous report (Ma and Mitra, 2000). To help you further understand these constructs, the Constructs pCATas was identical to pCAT and except that pCAT was in the sense orientation whereas pCATas was in the antisense orientation. Constructs pC3/NP and pC3/NPas were identical to pC3 and pC3 as except that promoters were removed in /NP constructs.

### Enzyme Assays

All GUS staining procedures were as described by Jefferson (1987). CAT assays were as described by Gorman et al. (1982).

### Molecular Characterization

RNA isolation and Northern blot analyses were carried out as described previously. To detect siRNA molecules, 50-60 μg RNA for each sample was fractionated on either a 15% TBE-PAGE gel with 7.0 M urea (Bio-Rad) or a 2.5% agarose gel. Fractionated RNA was transferred onto Zeta membrane (Bio-Rad) and probed by hybridization as described by the vendor except that hybridation was done at 50° C. for 36 hours.

TABLE 1
Table 1. Percentage of transgene silencing in primary transformants

| | Construct of the | Silencing frequency (%) | Number of |
|---|---|---|---|
| Promoter | transcriptional region | in primary transformants | plants tested |
| | CAT (pCAT) | 0 | 8 |
| | CAT/2A/GUS (pCAG) | 12 | 44 |
| | CAT/2A/CAT (pCAC) | 84 | 38 |
| | CAT/CAT (pCC) | 86 | 15 |
| | GUS/2A/GUS (pGAG) | 88 | 52 |
| | GUS/GUS (pGG) | 100 | 38 |
| | CAT/2A/GUS/2A/CAT (pCGC) | 82 | 34 |
| Enhanced CaMV | CAT/2A/CAT/2A/CAT (pC3) | 100 | 16 |
| 35S Promoter | CAT/2A/CAT/2A/CAT/2A/GUS (pC3G) | 96 | 26 |
| | GUS/2A/CAT/2A/CAT/2A/CAT (pGC3) | 100 | 34 |
| | CAT/2A/CAT/2A/CAT/2A/CAT (pC4) | 100 | 39 |
| | as[GUS/GUS] (pGGas)* | 100 | 15 |
| | as[CAT/2A/CAT/2A/CAT] (pC3as)* | 100 | 15 |
| | as[CAT/2A/CAT/2A/CAT/2A/CAT] (pC4as)* | 100 | 17 |
| | GFP (pGFP1) | 16 | 38 |
| | GFP/GFP/GFP (pGFP3) | 100 | 51 |
| | CAB1 (pCAB1) | 7 | 31 |
| | CAB1/CAB1/CAB1 (pB3) | 100 | 36 |
| Arabidopsis Chlorophyll | CAT (pPcab2/CAT) | 0 | 42 |
| a/b Binding Protein (cab2) | CAT/2CAT/2A/CAT/2A/CAT (pPcab2/C3) | 97 | 45 |
| Promoter | CAT/2A/CAT/2A/CAT/2A/CAT (pPcab2/C4) | 97 | 40 |

TABLE 2
Table 2. Segregation ration of non-silencing(NS)/silencing(S)
plants in individuals of T₁ progeny

| | Silencing in | | |
|---|---|---|---|
| Transgenic | (T₀) transgenic | No. of T₁ plants | NS/S ratio |
| lines | plant | tested | in T₁ progeny |
| C3-1 | + | 18 | 0/18 |
| C3-3 | + | 18 | 0/18 |
| C3-12 | + | 18 | 0/18 |
| C4-1 | + | 18 | 0/18 |
| C4-11 | + | 18 | 0/18 |
| C4-16 | + | 18 | 0/18 |
| C4-24 | + | 18 | 0/18 |
| CAC-8 | + | 18 | 12/6 |
| CAC-4 | − | 18 | 9/9 |
| CAT-1 | − | 18 | 18/0 |
| GUS-1 | − | 40 | 40/0 |

What is claimed is:

1. A method for suppressing the expression of a gene in a living cell, comprising:
a. identifying a target genetic sequence;
b. producing at least one copy of said target genetic sequence;
c. ligating said at least one copy of said target genetic sequence into a vector under the control of a promoter; and
d. incorporating said vector into said living cell.

2. A method for functional counterselection of living cells, comprising:
a. identifying a target genetic sequence;
b. producing at least one copy of said target genetic sequence;
c. ligating said at least one copy of said target genetic sequence into a vector under the control of a promoter;
d. incorporating said vector into said living cell;
e. placing said living cell in an environment capable of sustaining growth and multiplication; and
f. selecting progeny of said living cell displaying phenotypic traits consistent with suppression of the gene targeted by said target genetic sequence.

3. A method of identifying homology between different genes in a living cell, comprising:
a. identifying a target genetic sequence;
b. producing at least one copy of said target genetic sequence;
c. ligating said at least one copy of said target genetic sequence into a vector under the control of a promoter;
d. incorporating said vector into said living cell; and
e. observing the phenotypic change in said living cell, whereby said phenotypic change results from the loss of function of a related endogenous gene to said target genetic sequence, thereby identifying said related endogenous gene.
